(19) United States
(12) Patent Application Publication
(10) Pub. No.: US 2021/0233668 A1
(43) Pub. Date: Jul. 29, 2021

(54) TELE-PSYCHIATRY SYSTEMS AND METHODS

(71) Applicant: Health and Psychiatrist Consultants, LLC, Oldsmar, FL (US)

(72) Inventor: Usman Ezad, Oldsmar, FL (US)

(21) Appl. No.: 16/776,419

(22) Filed: Jan. 29, 2020

(51) Int. Cl. G16H 80/00; G16H 40/67; G16H 10/65; H04L 29/08; H04L 29/06; G06Q 10/10

(52) U.S. Cl. CPC G16H 80/00; G16H 40/67; G16H 10/65; G06Q 10/1095; H04L 67/104; H04L 67/04; H04L 63/083; H04L 67/12

(57) ABSTRACT

The invention relates to systems and methods for providing tele-psychiatry services. The systems and methods generally use a tele-psychiatry cart, a provider station and a secure network connection using a cloud-based, peer to peer, or hybrid network connection.

### BACKGROUND OF THE INVENTION

### 1) Field of the Invention

The present invention relates to tele-psychiatry, and more particularly to, systems and methods for secure transmission of information during a tele-psychiatry session.

### 2) Description of Related Art

Tele-psychiatry describes the use of telecommunication and information technology to provide psychiatry services. Tele-psychiatry may be used to provide improved access to psychiatry services in distant rural communities where psychiatry services may not be consistently available. Tele-psychiatry may also be used to address emergency situations at remote locations and/or in remote clinics, which lack psychiatry services.

During the use of a Tele-psychiatry system, there may be many transactions between a patient and a mental health care professional. For example, the telemedicine device may be used to upload the patient's private medical data over a communication network to a remote user, such as a psychiatrist. The psychiatrist may observe the patient over the video call and may analyze the patient's private medical data at a remote terminal.

However, transferring the patient's private medical data over the communication network, such as the internet, for example, may present opportunities for rogue interception and rogue use of the patient's private medical data. Furthermore, these communications may not be complaint with the Health Insurance Portability and Accountability Act of 1996 (‘HIPAA’). Thus, it may be desirable to have systems and methods for securely sharing a patient's private medical data over a communication link with a remote user.

Therefore, what is needed in the art is a tele-psychiatry system and method that provides a complaint and secure process for the sharing of a patient's private medical data.

### BRIEF SUMMARY OF THE INVENTION

The invention in one form is directed to a system for providing a secure communication between a client and a provider. The system including a tele-psychiatry station having a base, a motion apparatus configured to allow for movement of the tele-psychiatry station, a first extending member extending generally perpendicular to a top surface of the base and a mounting track. A plurality of mounts are attached to the mounting track. At least one output device is attached to at least one of the plurality of mounts. At least one input device is attached to at least one of the plurality of mounts. A client processor, having a client gateway configured for connecting to a provider gateway, is attached to the first extending member. A communication hub, used to connect the client processor to the at least one output device and the at least one input device, is attached to the first extending member. A power distribution hub, providing power to at least one of the at least one output device, the at least one input device and the client processor, the power distribution hub being, is attached to the at least one extending member.

The system further includes a provider station having a provider processor with a provider gateway configured for connecting to the client gateway. A provider output device and a provider input device are connected to the provider processor. A connection is used between the tele-psychiatry station and the provider station. The peer to peer network connection uses a software that resides on at least one of the client processor and the provider processor to facilitate the connection.

The invention in another form is a method of using a tele-psychiatry station for providing a remote psychiatry service. The method includes the steps of providing a tele-psychiatry station. The tele-psychiatry station includes a base, a motion apparatus configured to allow for movement of the tele-psychiatry station, and a first extending member extending generally perpendicular to a top surface of the base. The first extending member further including a mounting track. A plurality of mounts attach to the mounting track with at least one output device being attached to at least one of the plurality of mounts. At least one input device is also attached to at least one of the plurality of mounts. A client processor having a client gateway configured for connecting to a provider gateway is attached to the first extending member. A communication hub connecting the client processor to the at least one output device and the at least one input device is attached to the first extending member. A power distribution hub providing power to at least one of the at least one output device, the at least one input device and the client processor is attached to the at least one extending member.

The method further includes a step for providing a provider station having a provider processor with a provider gateway that is configured for connecting to the client gateway. The provider station also includes a provider output device and a provider input device. Another step of the method provides for opening a connection between the tele-psychiatry station and the provider station. The connection uses a software residing on at least one of the client processor and the provider processor to facilitate the peer to peer network connection. Using the peer to peer connection, an interactive communication between a user using the tele-psychiatry station and a provider using the provider station occurs. The interactive communication includes at least one of a psychiatric evaluation, a patient education and a medication management.

In yet another form, the invention is a method for providing a remote psychiatry service. The method includes steps for providing a facility with a client processor, providing a provider with a provider processor and creating an appointment between the facility, the provider and a client. In other steps, the method is validating at least one information of the client prior to starting the appointment, providing a secure location within the facility for the client to use the client processor, and establishing a connection between the client processor and the provider processor. The method includes a step for using a software to allow an interactive communication between the client processor and the provider processor. The method further includes using the interactive communication to provide at least one of a psychiatric evaluation, a patient education and a medication management.

An advantage of the present invention is a secure HIPAA complaint communication.

Another advantage of the present invention remote psychiatry service for patients in areas underserved with mental health services.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1a shows an embodiment of the present invention for providing a tele-psychiatry service;

FIG. 1b shows another view of the embodiment shown in FIG. 1a;

FIG. 2 shows another embodiment of the present invention for providing a tele-psychiatry service;

FIG. 3 shows one method of an embodiment of the present invention; and

FIG. 4 shows another method of an embodiment of the present invention; and

FIG. 5 shows a block diagram of one embodiment of the invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

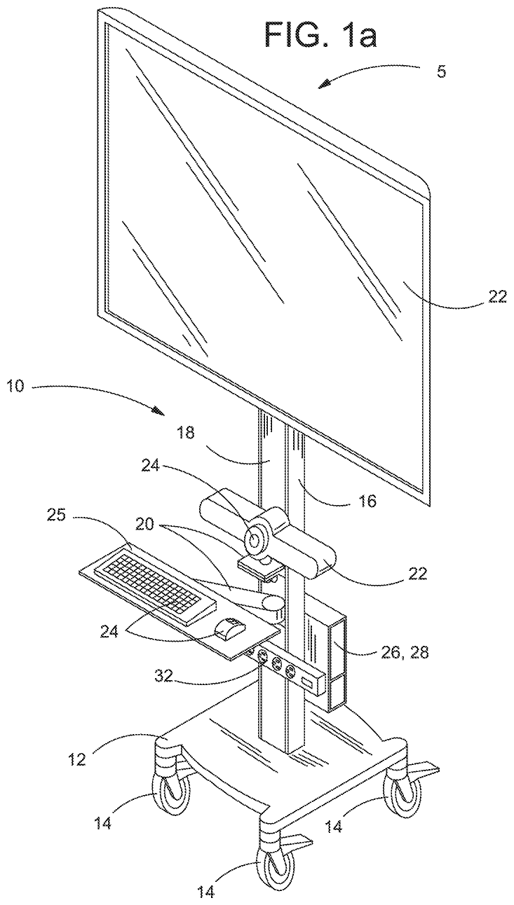

Referring now to the drawings, and more particularly to FIGS. 1a and 1b, there is shown part of a system 5 for providing a remote psychiatry service using a secure communication between a client, e.g., patient, receiver of mental health services, and a provider, e.g., psychiatrist, physician assistant-certified (‘PA-C’), advanced registered nurse practitioner (‘ARNP’). A secure communication between the client and the provider is defined as a communication of data that is transmitted across a network, e.g., cloud based and peer to peer (‘P2P’), and protected by encrypting with Advanced Encryption Standard (AES), i.e., AES-128 for 128-bit encryption or AES-256 for 256-bit encryption, for any meeting data, e.g., audio and video. Any stored data, e.g., medical files, transmitted may be protected using a message authentication code (‘MAC’) such as hash-based message authentication code (‘HMAC’) SHA-256 or SHA-3. A key may be generated and securely distributed to all participants at the start of each session to access the data distributed during the communication.

A remote psychiatry service, i.e., meeting or session, is generally between a client and a provider. Access control to the meeting may be controlled through a verification of email addresses and passwords. The host of the meeting, typically the provider, can control when the session is terminated. The system may also include a preset to automatically terminate the session.

The system includes a tele-psychiatry station 10 used to facilitate the secure communication between the client and the provider and is generally configured for use by the client. The tele-psychiatry station 10 may further include a base 12 having a top and a bottom. The base 12 is designed to provide a supporting platform for the tele-psychiatry station 10 and may further include a motion apparatus 14 attached to the bottom of the base 12. The motion apparatus 14 is designed to allow for movement of the tele-psychiatry station 10 from one location to another. The motion apparatus 14 may be any suitable device used for movement, such as castors, wheels, tracks and/or discs made of materials having a low coefficient of static friction, e.g., polytetrafluoroethylene (‘PTFE’).

The tele-psychiatry station 10 further includes a first extending member 16 extending generally perpendicular to the top surface of the base 10. The first extending member 16 may have any length and is configured to provide a generally vertical support. The first extending member 16 may further include a mounting track 18 extending along a length of the first extending member 16. The mounting track 18 may be formed integral to the first extending member 16 or it may be attached to the first extending member 16. Attachment of the mounting track 18 may be completed through non-removable methods, e.g., welding, adhesive, or attachment may be completed through removable methods, e.g., bolting, pins and clamps. The mounting track 18 may be provided as a channel, e.g., c-channel, to allow for positioning at any point along the channel or the mounting track 18 may be provided as discrete mounting points along its length.

The tele-psychiatry station 10 may further include a plurality of mounts 20 that are attached to the mounting track 18. The attachment of the mounts 20 may be at a predetermined location or the mounts 20 may be slidably adjustable along the length of the mounting track 18. Providing the mounts 20 with a slidable adjustment advantageously provides infinite height adjustments along the length of the mounting track 18 to suit height and preference differences between multiple clients.

The mounts 20 provide locations to attach devices to the tele-psychiatry station 10. The devices may include video monitors, speakers, microphones and cameras. The mounts 20 may provide a rigid connection between the device and the tele-psychiatry station 10 or the mounts 20 may include a mechanical joint, e.g., knuckle, turnbuckle, pin, cotter, bolted, screw, ball, and prismatic, to allow for articulation of the device relative to the tele-psychiatry station 10.

Multiple devices may be added to the tele-psychiatry station 10 such as an output device 22 and an input device 24. As discussed above, the output device 22 and the input device 24 may be attached to the tele-psychiatry station 10 via a mount 20. It should also be considered that the output device 22 and the input device 24 may be attached directly to the extending member 16.

An output device 22 may be any type of device used by a client to receive a communication from the tele-psychiatry station 10 such as an audio or visual communication from the provider or facility, or from a file stored on the system. Typical devices suitable for an output device include a video monitor, a printer, display panel, speaker, refreshable braille display or another device capable of producing visible, audible, or tactile output.

An input device 24 may be any type of device used by a client to send a communication from the tele-psychiatry station 10 to the provider or facility, or to a file stored on the system. Typical devices suitable for an input device include a keyboard, keypad, a pointing device, a camera, a microphone and a refreshable braille display. The tele-psychiatry station 10 may also include a platform 25 that may be attached to the first extending member 16. The platform 25 may be used to support any of the output device 22 and input device 24. It is to be understood that the tele-psychiatry station 10 may include any number of devices to facilitate communication, such as multiple video monitors used to display a provider and a file, multiple speakers, and multiple cameras to provide alternate viewing angles.

The tele-psychiatry station 10 also includes a client processor 26. The client processor 26 may include one or more processing units, e.g. one or more computers. Client processor 26 may be configured to operate in accordance with programmed instructions stored in a memory. Client processor 26 may be capable of executing an application for sharing personal data stored in a memory on the tele-psychiatry station 10 with a remote user, e.g., provider, mental health care provider.

In some embodiments of the invention, the personal data referred to herein may include any private and/or confidential medical data of the user of tele-psychiatry station 10 and/or records of any transactions that occurred by any user using tele-psychiatry station 10. Records of any transactions that occurred by any user using tele-psychiatry station 10 may include a log of calls with dates, times, the identification of the remote user such as a clinic and/or a doctor who was connected to the user, e.g., the client or patient. The personal data may be encrypted and sent to the remote user, e.g., a provider, at the remote terminal for viewing.

However, the tele-psychiatry station 10 may also store secure personal data related to the user of tele-psychiatry station 10. Secure personal data in the context of this patent application may include, for example, protected health information data and/or personally identifiable information data. Protected health information data may include individually identifiable health information including demographic data, such as the user's past, present or future physical or mental health or condition, the administration of health care to the user, payments made to administering health care to the user, and/or the user's identity. Protected health information data may include information which can be, used to distinguish or trace the user's identity, such as the user's name, social security number, biometric records, date and place of birth, mother's maiden name, driver's license number, account numbers, credit or debit card numbers, and/or any information providing access to the user's financial account such, access codes and/or passwords.

In some embodiments of the invention, client processor 26 may be configured to prevent the secure personal data of the telemedicine device user stored on tele-psychiatry station 10 from being sent to provider processor 36. In some embodiments, for example, client processor 26 may prevent the secure personal data from being sent out of the tele-psychiatry station 10 by encrypting the secure personal data with strong encryption using a private key of the tele-psychiatry station 10, which may be placed in the key storage of the tele-psychiatry station 10. Thus, even if a rogue user does manage to intercept the secure personal data stored on the tele-psychiatry station 10, the rogue user will be unable to decrypt the secure personal data. In some embodiments of the present invention, client processor 26 may be configured to erase protected health information data of the user after each session.

Client processor 26 may communicate with output device 22. For example, output device 22 may include a computer monitor or screen. Client processor 26 may communicate with a screen of output device 22 to display information for the tele-psychiatry station 10 user. In another example, output device 22 may include a video monitor, a printer, display panel, speaker, refreshable braille display or another device capable of producing visible, audible, or tactile output.

In some embodiments, the tele-psychiatry station 10 may include a light emitting diode (‘LED’) display used for interaction with the provider. It is to be understood that the display may be of any size suitable for each facility. Generally, the LED monitor is sized from 19″ to 80″. Typically, a 64″ LED is used for interaction with the provider.

Client processor 26 may communicate with input device 24. For example, input device 24 may include one or more of a keyboard, keypad, a pointing device, a camera, a microphone and a refreshable braille display for enabling a user to inputting data or instructions for operation of client processor 26. A touch screen, for example, may be used to provide functionality of both an input device 24 and an output device 22.

Client processor 26 may communicate with a memory. Memory may include one or more volatile or nonvolatile memory devices. Memory may be utilized to store, for example, programmed instructions for operation of client processor 26, data or parameters for use by client processor 26 during operation or results of operation of client processor 26.

Memory may include a computer readable medium for storing program instructions for operation of client processor 26. It is noted that memory and/or any suitable data storage device communicating with client processor 26 may be remote from client processor 26. Memory may store a module in the form of an installation package or packages that can be downloaded and installed for execution by client processor 26. Memory may be utilized to store data or parameters for by client processor 26 during operation, or results of operation of client processor 26.

In operation, client processor 26 may execute a method for sharing personal data stored in memory on the tele-psychiatry station 10 or provide an interactive communication with a remote user using a client gateway 28. The client processor 26 may be permanently or removably attached to the first extending member 16.

The client gateway 28 may be any type of gateway suitable for transmission of audio and/or video data.

The tele-psychiatry station 10 may also include a communication hub 30 and a power distribution hub 32. The communication hub 30 is configured for connecting the client processor 26 to the output device 22 and the input device 24. The communication hub 30 may be permanently or removably attached to the first extending member 16. The communication hub 30 may include any type of connection suitable for connecting devices to a client processor 26, such as, Video Graphics Array (‘VGA’), Digital Visual Interface (‘DVI’), Display Port, Mini-Display Port, High Definition Multimedia Interface (‘HDMI’), Mini High Definition Multimedia Interface (‘Mini-HDMI’), Micro High Definition Multimedia Interface (‘Micro-HDMI’), Personal System/2 (′PS/2′), Firewire, Serial Advanced Technology Attachment (‘SATA’), ethernet, 3.5 mm audio cable, Universal Serial Bus (′USB-A′, ‘USB-B’, ‘USB-C’, ‘USB 3’), Mini Universal Serial Bus (‘Mini-USB’), Micro Universal Serial Bus (‘Micro-USB’), Universal Serial Bus 3.0 (′USB 3.0′, ‘USB 3.0 Micro’).

The power distribution hub 32 may provide power to any of the devices on the tele-psychiatry station 10. In general, power distribution hub 32 provides a 120V electrical source at 12A with a resettable surge protector. The power distribution hub 32 may provide power to an output device 22, an input device 24 and the client processor 26. The power distribution hub 32 may be permanently or removably attached to the first extending member 16.

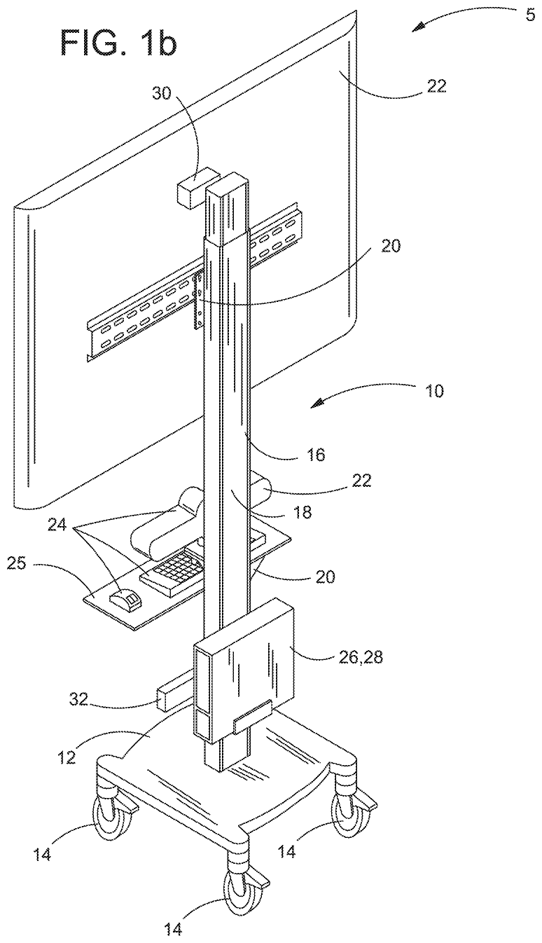

Referring now to FIG. 2, there is shown another part of system 5 for providing a secure communication between a client and a provider which includes a provider station 34 configured for use by a provider. The provider station 34 includes a provider processor 36 with a provider gateway 38 configured for connecting to the client gateway 28. The provider processor 36 may include one or more processing units, e.g. one or more computers. Provider processor 36 may be configured to operate in accordance with programmed instructions stored in a memory. Provider processor 36 may be capable of executing an application for sharing personal data stored in a memory on the provider station 34 with a remote user, e.g., client.

In some embodiments of the present invention, if properly authorized, provider station 34 may communicate personal data and/or records stored in memory, for example, with an electronic medical record (‘EMR’) system or to send a pharmacy a prescription for the client. The provider station 34 may communicate with the EMR system or pharmacy through a second connection 46 or the provider station 34 may use the first connection 44 if using a cloud-based server. It is to be understood that the second connection 46, if utilized, will include the same security features as the first connection 44.

The provider gateway 38 may be any type of gateway suitable for transmission of audio and/or video data over a network.

Provider processor 36 may communicate with provider output device 40. For example, provider output device 40 may include one or multiple computer monitors or screens. Provider processor 36 may communicate with a screen of the provider output device 40 to display information for the provider station 34 user. In another example, provider output device 40 may include a video monitor, a printer, display panel, speaker, refreshable braille display or another device capable of producing visible, audible, or tactile output.

In some embodiments, the provider station 34 may include two output devices 40, e.g., light emitting diode (‘LED’) displays. The LED displays may include one for interaction with the client and the other for data entry by the provider into an electronic medical record. It is to be understood that the displays may be of any size suitable for each provider. Generally, the LED monitors are sized from 19″ to 64″. Typically, a 32″ LED is used for entry of information into the electronic medical record of the client and 45″ for interaction with the client.

Provider processor 36 may communicate with provider input device 42. For example, provider input device 42 may include one or more of a keyboard, keypad, a pointing device, a camera, a microphone and a refreshable braille display for enabling a user to inputting data or instructions for operation of provider processor 36. A touch screen, for example, may be used to provide functionality of both a provider input device 42 and a provider output device 40.

Provider processor 36 may communicate with a memory. Memory may include one or more volatile or nonvolatile memory devices. Memory may be utilized to store, for example, programmed instructions for operation of provider processor 36, data or parameters for use by provider processor 36 during operation or results of operation of provider processor 36.

Memory may include a computer readable medium for storing program instructions for operation of provider processor 36. It is noted that memory and/or any suitable data storage device communicating with provider processor 36 may be remote from provider processor 36. Memory may store a module in the form of an installation package or packages that can be downloaded and installed for execution by provider processor 36. Memory may be utilized to store data or parameters for by provider processor 36 during operation, or results of operation of provider processor 36.

In operation, provider processor 36 may execute a method for sharing personal data stored in memory on the provider station 34 or provide an interactive communication, e.g., psychiatry service, with a remote user using a provider gateway 38.

The system 5 for providing a secure communication between a client and a provider also includes a first connection 44 between the tele-psychiatry station 10 and the provider station 34. The first connection 44 may be a peer to peer network connection or it may be a cloud-based network connection.

A peer to peer network (‘P2P’), is a type of network in which the processors or computers are connected to each other directly via the Internet. Files can be shared directly between systems on the network without the need of a central server. In other words, each computer on a P2P network becomes a file server as well as a client.

A cloud-based network is a type of network that uses a central server to connect each of the processors or computers of the network together. The central server of the cloud-based network receives data from a file server and then sends that data to a client. Each computer on a cloud-based server may also become a file server and a client. Generally, the cloud-based network provides flexibility for adding additional computing systems to the network by connecting all computers to one central hub. Therefore, in a P2P, any meeting data or stored data is transferred directly from one computer to another; whereas, in a cloud-based network, any data is transferred from one computer to a central server and then to the next computer.

In the present invention, it is to be understood that the first connection 44 may be a P2P, a cloud-based or a hybrid of the two. In a hybrid configuration, the cloud-based network may initiate a ‘hand-shake’ between the two computers and then exit the first connection 44 when the ‘hand-shake’ is complete. Generally, the ‘hand-shake’ will involve a validation of the emails and passwords of the computers prior to allowing a P2P connection from being established.

The first connection 44 uses a software that resides on at least one of the client processor 26, the provider processor 36 and/or a cloud-based server. The software may include features to control access to the first connection 44 such as, encrypting all meeting data using an encryption method such as Advanced Encryption Standard (AES), i.e., AES-128, AES-192 or AES-256, verifying an email address and password, allowing the provider to terminate access to the meeting or terminate the meeting, and/or automatically terminating a meeting that has been paused for a predetermined amount of time. The software may also protect the integrity of any stored data shared with a protection method such as hash-based message authentication code (‘HMAC’) SHA-256 or SHA-3. A key may be generated and securely distributed to all participants at the start of each session to access the data distributed during the communication.

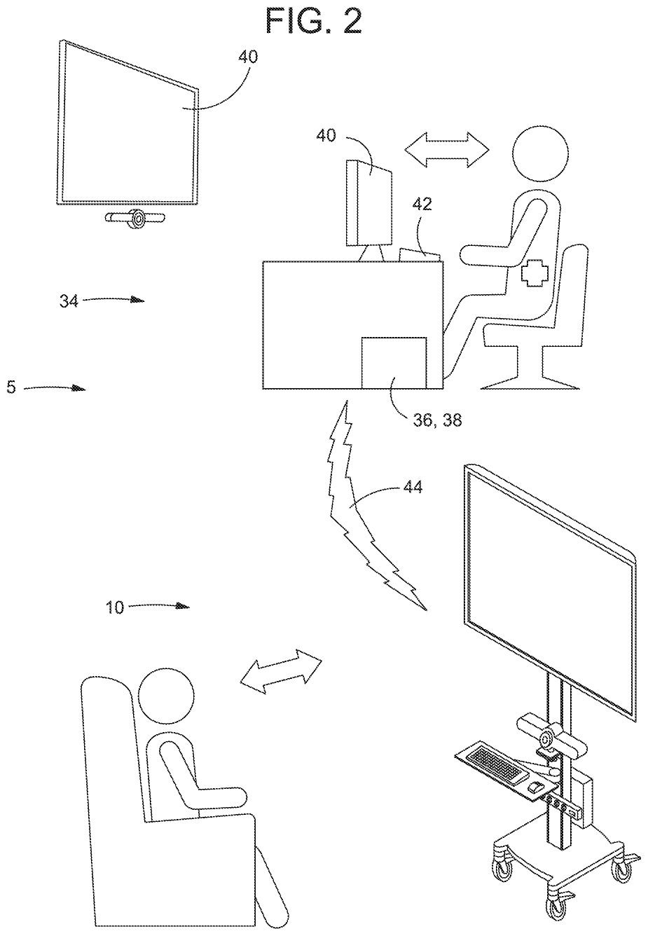

Referring now to FIG. 3, there is shown a method 100 for using a tele-psychiatry station 10 to provide remote psychiatry services. The method 100 generally includes a step 110 for providing a tele-psychiatry station 10 as described in the proceeding paragraphs. A step 112 for providing a provider station 18 as described in the proceeding paragraphs. A step 114 for opening a connection 44 between the tele-psychiatry station 10 and the provider station 18. The connection 44 using a health insurance portability and accountability act complaint software residing on at least one of the client processor 26 and the provider processor 36 to facilitate the connection. A step 116 for having an interactive communication, e.g., psychiatry service, between a client using the tele-psychiatry station 10 and a provider using the provider station 18. The interactive communication to include at least one of a psychiatric evaluation, a patient education and a medication management.

In another embodiment of the method, the method may include steps allowing a client to find a facility near them and set an appointment. When setting the appointment, the client may log into a secure portal showing available times, log in through a mobile application, select a time on a website, or call in to the facility. When the client arrives for their appointment, the client fills out initial new patient paperwork. Paperwork may be filled out on paper and then manually entered into a database or the client may fill out an electronic form for automatic entry in the database. Medical staff will then scan the client's driver license and insurance card. Client will also pay co-pay if one is required. Medical staff will walk the client to a private room. Client is then seated in a comfortable environment. Typically, the client will have a one-on-one session with the provider. Typical sessions with the provider have a duration of one hour. However, the duration may be more or less as dictated by the needs of the client and available time of the provider. The one-on-one session is conducted via a 100% HIPAA compliant tele-psychiatry cart. After the one-on-one session, the provider may send a prescription to the client's preferred pharmacy via an EMR software. Client may then set up a follow up appointment with medical staff.

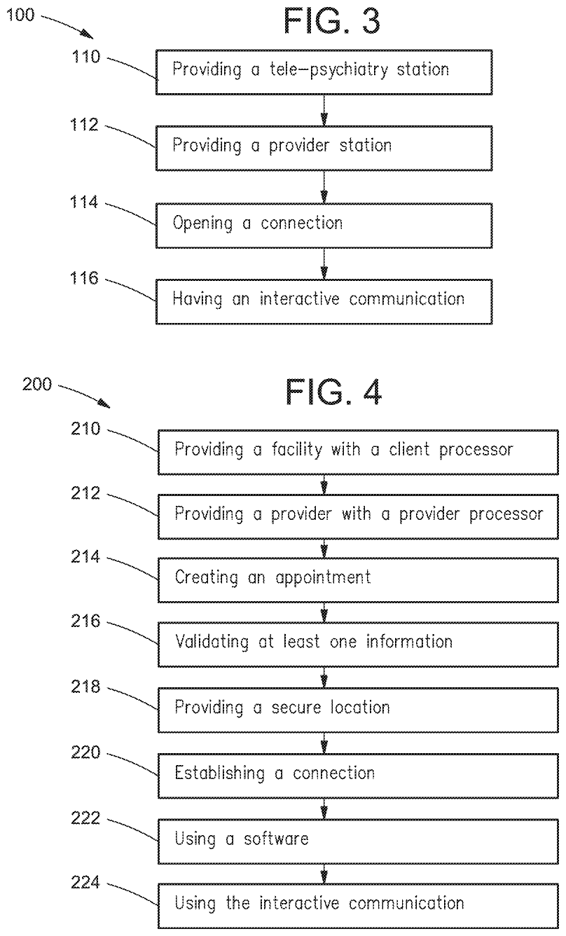

Referring now to FIG. 4, there is shown a method 200 for providing a remote psychiatry service. The method 200 generally including the step 210 of providing a facility with a client processor 26. A facility may be any type of location suitable for providing mental health services, such as, a hospital, a doctor office, or a mental health care provider. A step 212 for providing a provider with a provider processor 36. A step 214 for creating an appointment between the facility, the provider and a client. An appointment may be any suitable time of the day for conducting the remote psychiatry services. A step 216 for validating at least one information of the client prior to starting the appointment. The information of the client may be any type of information, including protected health information data and/or personally identifiable information data. A step 218 for providing a secure location, e.g., a location where the client will have privacy from the rest of the facility such as a private room, within the facility for the client to use the client processor 26. A step 220 for establishing a connection 44 between the client processor 26 and the provider processor 36. A step 222 for using a software to allow an interactive communication between the client processor 26 and the provider processor 36. A step 224 for using the interactive communication to provide at least one of a psychiatric evaluation, a patient education and a medication management.

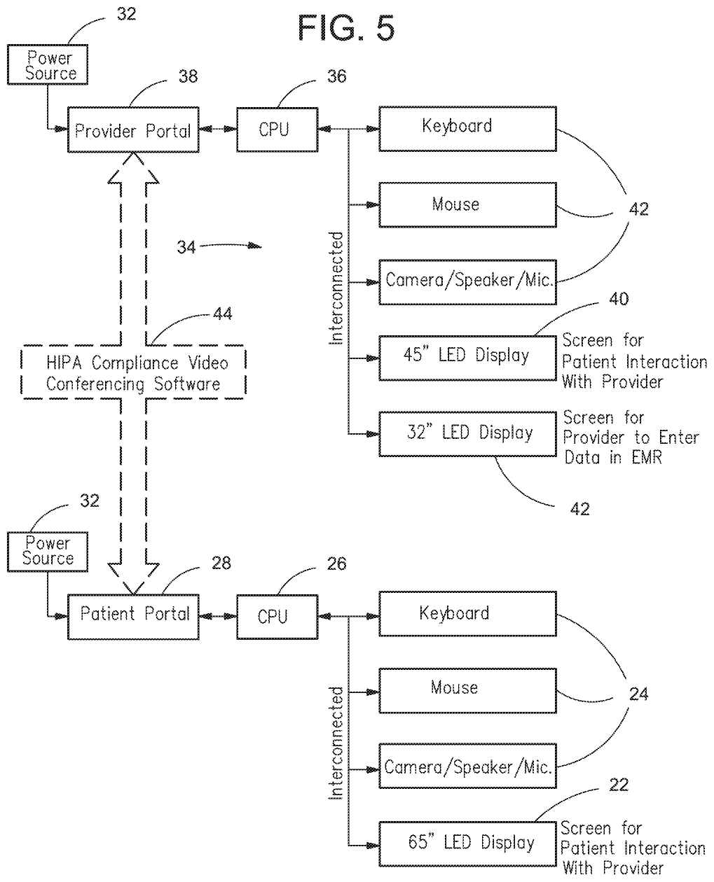

Referring now to FIG. 5 there is shown another embodiment of system 5 for providing a remote psychiatry service using a secure communication between a client, e.g., patient, receiver of mental health services, and a provider, e.g., psychiatrist, physician assistant-certified (‘PA-C’), advanced registered nurse practitioner (‘ARNP’). The embodiment includes a tele-psychiatry station 10 with client output devices 22, i.e., 65″ LED display and a speaker, and client input devices 24, i.e., camera, microphone, keyboard and mouse. The tele-psychiatry station 10 also includes a client processor 26, client gateway 28 and a power distribution hub 32.

The embodiment also includes a provider station 34 having provider output devices 40, i.e., 32″ LED display, 45″ LED display, and speaker. The provider station 34 also includes provider input devices 42, i.e., keyboard, mouse, camera and microphone. The provider station 34 further includes a provider processor 35, a provider gateway 38 and a power distribution hub 32. The tele-psychiatry station 10 and the provider station 34 are connected with a secure connection 44. The secure connection 44 may be one of a peer to peer network or a cloud-based network connection.

Some embodiments of the present invention may include an article such as a computer or processor readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical, e.g., electronic, quantities within the computer's registers and/or memories into other data similarly represented as physical quantities within the computer's registers and/or memories or other information non-transitory storage medium, e.g., a memory, that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive, any or all of the stated options.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for providing a secure communication between a client and a provider, the system comprising:
a tele-psychiatry station including:
a base;
a motion apparatus configured to allow for movement of the tele-psychiatry station;
a first extending member extending generally perpendicular to a top surface of the base, the first extending member further including a mounting track;
a plurality of mounts attaching to the mounting track;
at least one output device being attached to at least one of the plurality of mounts;
at least one input device being attached to at least one of the plurality of mounts;
a client processor having a client gateway configured for connecting to a provider gateway, the client processor being attached to the first extending member;
a communication hub connecting the client processor to the at least one output device and the at least one input device, the communication hub being attached to the first extending member;
a power distribution hub providing power to at least one of the at least one output device, the at least one input device and the client processor, wherein the power distribution hub is attached to the at least one extending member;
a provider station including:
a provider processor having the provider gateway configured for connecting to the client gateway;
a provider output device in connection with the provider processor;
a provider input device in connection with the provider processor;
a first connection between the tele-psychiatry station and the provider station, the first connection is at least one of a peer to peer network and a cloud-based network to facilitate the first connection; and
a software residing on at least one of the client processor, the provider processor and a cloud-based server.

2. The system of claim 1, wherein the first connection is established using the cloud-based network having the software, wherein the software requires a unique email address and a unique password to allow for the transfer of a data over the first connection.

3. The system of claim 2, wherein the first connection once established by the cloud-based server is converted to a peer to peer network between the tele-psychiatry station and the provider station.

4. The system of claim 1, wherein the provider station includes a second connection configured for transferring a data with at least one of electronic medical record system and a pharmacy.

5. A method of using a tele-psychiatry station for providing a remote psychiatry service, the method comprising the steps of:
providing the tele-psychiatry station having a base, a motion apparatus configured to allow for movement of the tele-psychiatry station, a first extending member extending generally perpendicular to a top surface of the base, the first extending member further including a mounting track, a plurality of mounts attaching to the mounting track, at least one output device being attached to at least one of the plurality of mounts, at least one input device being attached to at least one of the plurality of mounts, a client processor having a client gateway configured for connecting to a provider gateway, the client processor being attached to the first extending member, a communication hub connecting the client processor to the at least one output device and the at least one input device, the communication hub being attached to the first extending member, and a power distribution hub providing power to at least one of the at least one output device, the at least one input device and the client processor, the power distribution hub being attached to the at least one extending member;
providing a provider station having a provider processor with the provider gateway configured for connecting to the client gateway, a provider output device, and a provider input device;
opening a first connection between the tele-psychiatry station and the provider station, the first connection using a software residing on at least one of the client processor and the provider processor to facilitate the first connection; and
having an interactive communication between a client using the tele-psychiatry station and a provider using the provider station, the interactive communication to include at least one of a psychiatric evaluation, a patient education and a medication management.

6. The method of claim 5, wherein the first connection is established using the cloud-based network having the software, wherein the software requires a unique email address and a unique password to allow for the transfer of a data over the first connection.

7. The method of claim 6, wherein the first connection once established by the cloud-based server is converted to a peer to peer network between the tele-psychiatry station and the provider station.

8. The method of claim 5, wherein the provider station includes a second connection configured for transferring a data with at least one of electronic medical record system and a pharmacy.

9. A method for providing a remote psychiatry service, the method comprising the steps of:
providing a facility with a client processor;
providing a provider with a provider processor;
creating an appointment between the facility, the provider and a client;
validating at least one information of the client prior to starting the appointment;
providing a secure location within the facility for the client to use the client processor;
establishing a first connection between the client processor and the provider processor;
using a software to allow an interactive communication between the client processor and the provider processor; and
using the interactive communication to provide at least one of a psychiatric evaluation, a patient education and a medication management.

10. The method of claim 9, wherein establishing the first connection further includes steps of using a cloud-based server to validate the client processor and the provider processor, securing the connection between the client processor and the provider processor, and allowing a meeting data and a stored data to transfer directly between the client processor and the provider processor.

11. The method of claim 9, wherein the first connection is a peer to peer network connection and the method includes a step for establishing a second connection configured for sending a data to at least one of an electronic medical record and a pharmacy.

12. The method of claim 11, wherein the software requires a unique email address and a unique password to allow for the transfer of a data over the first connection and the second connection.

13. The method of claim 11, wherein in the second connection to the pharmacy is used to transmit a prescription.

14. The method of claim 9, wherein the provider processor includes a memory for facilitating the interactive communication.
